(19) United States
(12) Patent Application Publication
(10) Pub. No.: US 2010/0074926 A1
(43) Pub. Date: Mar. 25, 2010

(54) MODULES, NEW ASSEMBLAGE KITS AND NEW ASSEMBLES FOR THE CONTROLLED RELEASE OF SUBSTANCES

(75) Inventors: Paolo Colombo, Parma (IT); Patrizia Santi, Salsomaggiore Terme (IT); Ruggero Bettini, San Polo Di Torrile (IT); Orazio Luca Strusi, Parma (IT); Fabio Sonvico, Cadorago (IT); Gaia Colombo, Ferrara (IT)

Correspondence Address: COLLARD & ROE, P.C., 1077 NORTHERN BOULEVARD, ROSLYN, NY, 11576, US

(73) Assignee: UNIVERSITA' DEGLI STUDI DI PARMA

(21) Appl. No.: 12/312,831

(22) PCT Filed: Dec. 5, 2006
(86) PCT No.: PCT/EP2006/011661

§ 371 Date: May 28, 2009

(51) Int. Cl. A61K 9/00; A61J 3/10

(52) U.S. Cl. 424/400

(57) ABSTRACT

A module which is destroyed in the presence of water solution, composed of a compressed powdery mixture, the said powdery mixture comprising a matrix building component, suitable to release an optionally included active substance into a surrounding aqueous liquid, and one or more tabletting aids chosen from the group consisting of lubricants, glidants, and anti-adherent agents, the said module being provided with particular male topological features allowing its connection to a corresponding female module, giving raise to an assembly that can be safely handled on industrial scale, wherein, in the composition of the male module, the percentage ratio between the tabletting aid and the matrix building component is comprised between 1:2.5 and 1:999. The module can be employed to convey pharmaceuticals, nutraceuticals, agrochemicals or other active principles to the intended site of action.

### FIELD OF THE INVENTION

The present invention concerns the field of pharmaceutical or nutraceutical or agrochemical formulations for the controlled release of an active substance. In the aforementioned fields, it is customary to formulate an active substance (which can be, e.g. a medicament, a vitamin, etc. a plant growth hormone, a pesticide, a herbicide, a fertilizer etc.) together with excipients which, on top of “diluting” the active principle and allowing the manufacturing of a dosage form and administration thereof to a human, or to an animal or plant, provide for the provision of a predetermined release profile of the said active substance to the physiologic or natural environment in time and space. It is peculiar to the physiologic or natural environments at issue, that these are characterized by the presence of at least one aqueous fluid, e.g. a physiological fluid, into which release of the active substance is desired.

### TECHNICAL BACKGROUND

As said above, nowadays, in a lot of applications spreading from human pharmaceutical use to the treatment of plants and animals it is essential to deliver to the organism to be treated active substances not only in the appropriate dosage form but also in a controlled and reproducible manner. Moreover, the increasing need to administer at the same time several substances has complicated the protocols of treatment to be followed e.g. by the health-care professionals. For example, in many pharmaceutical applications, it is necessary to adapt the release properties of the delivery system to special biological requirements. Some therapies that need particular timing and scheduling of drug administration are: the Parkinson disease treatment with levodopa and carbidopa combination, the chronoschedule of anti-inflammatory or anti-asthma drugs and careful dose adjustment with oral anticancer drugs. As a consequence, the innovations in the pharmaceutical domain have focused on the development of formulations able to provide flexibility in delivery, i.e., they should release the drug at different rates and sites according to a delivery program obtained by controlled modifications of their manufacturing conditions. Among the different types of formulations currently available, solid formulations are by far preferred form the industrial point of view, be it for their ease of manufacture and handling, but also for ease of administration and conveyance to the site of action. Among the solid formulations, those are largely preferred in which the excipients and active principle(s) which are co-formulated together, are provided in the form of compact bodies (e.g. matrices constituted of compressed powders) which may be suitably administered (e.g. taken by mouth in pharmaceutical or nutraceutical applications, or placed close to the treated plant in agricultural applications) and which thereafter undergo a controlled interaction with the physiological/natural environment, leading to release of the harbored active principle into the surrounding aqueous fluid.

One of the aspects investigated in the adjustment of active principle delivery profiles from such matrices into the aqueous fluid is, further to their composition, their geometrical shape. For example, to comply with the specific needs of controlled release of pharmaceutical substances, the so-called Geomatrix® technology (described in U.S. Pat. No. 4,839,177) has been developed by one of the present inventors, which consists in the provision of a pharmaceutical dosage form comprising two different phases, (a) a deposit-core phase harboring the active principle in a first mixture of excipients, comprising high loads of polymeric materials having swelling/and or gelling properties in contact with water or aqueous fluids, and (b) a support-platform phase, coated onto said deposit core, consisting of a polymeric material insoluble in aqueous liquids and partially covering said deposit core. With this technology, it this thus possible to influence the surface area/volume ratio of the deposit core of a given, predetermined shape and thus its delivery kinetics upon contact with the physiological fluid by appropriately designing the geometry of the support platform coated thereon. Although this method provided a reliable time control, it could not target the delivery site.

On top of that, with the Geomatrix® technology, application of the support platform of the desired shape was performed either by compression of the solid second phase onto the first phase or by immersion/spraying techniques employing a suitable solution of the second phase. However, both of these application techniques are laborious in industrial practice and bear the risk of spoiling or altering the first provided deposit core, which either undergoes high compression stress or must be suitably dried after the immersion/spray treatment. Moreover, the biphasic Geomatrix® matrices, once obtained according to the predefined design initially envisaged, cannot be further modified.

In view of the aforementioned drawbacks, the authors of the present patent application introduced recently a new, modular approach for preparing controlled release drug delivery systems characterized by higher flexibility and ease of industrial processing. The new technology was labeled <<release modules assemblage>> technology and is described in WO 03/043601. The release modules used in this assembly technology were solid structures obtained by powder compression (or other suitable method) of mixtures of excipients optionally comprising an active principle, giving rise to porous compacts with appropriate shape for rendering assemblage more easy. According to such strategy, two to several release modules allowed the construction of assemblies, which according to the number, nature and orientation of the single modules employed, allowed, according to the resulting overall topology thereby achieved, the matching of previously defined release kinetics into an aqueous fluid. The resulting assemblies were administered to the patients as such or after introduction into a suitable biocompatible outer containment such as, e.g. a hard gelatin capsule.

It goes without saying that the “release modules assemblage” technology, unlike the earlier Geomatrix® approach, further to allowing the achievement of a pre-established or customized release kinetics, also allows (i) for the pre-established or customized combination of different active principles or (ii) for the pre-established or customized dosage and release of a given active principle, namely when (i) different modules contain different active principles or when further active principles are included in hollow spaces circumscribed by the assemblies, or when (ii) modules of equal or different dosage are combined into assemblies characterized by different relative position.

The modules realized according to WO 03/043601 had cylindrical shape with one convex base bearing a dome-shaped protrusion and the other base concave, accommodating a complementary recession matching with the aforementioned dome-shaped protrusion, in order to ease assemblage of the modules.

According to the way of assemblage, namely concave/concave or concave/convex, different type of assemblies, including an internal hollow inner space or not, could be obtained.

In case non-adhesive polymers were employed for the construction of the modules and concave/convex assemblage according to WO 03/043601 was desired, the required stability of the assemblies in conditions of use was provided by gluing or ultrasound welding the pile of modules to make up the assembly. In the alternative, stability in conditions of use was provided by secondary interaction of the assemblies with the surrounding aqueous fluid encountered during drug release, e.g. by the gelling in the stomach/intestine of the polymers employed in the construction of the modules, such as to enhance their mutual engagement. Still as an alternative, stability in conditions of use was ensured by the formation of a sticky outer layer, tying the modules together and resulting from the initial decomposition of an outer gelatin shell enclosing the assembly, as a consequence of the contact with a physiological fluid.

On the contrary, in case of concave/concave alignment, gluing or welding was mandatory to obtain a sealed inner space.

However, the “release modules assemblage” technology is in the need of further improvement, as far as ease of industrial handling of the modules and of the assemblies obtained is concerned. This is because, whereas it is possible, through the means described above, to ensure proper fitting of the known modules in conditions of use, i.e. through interaction with an aqueous environment, a completely reliable fitting during upstream industrial handling is achievable only through additional processing steps, such as gluing or welding. This is because the employment of adhesive polymers in the formulation of the excipient mixture, while theoretically possible, is limited on the other hand, by the risk of producing, with growing intrinsic stickiness of the modules, non-properly oriented, “random” agglomerates of an excessive number of modules. Such “random” agglomerates form spontaneously during industrial handling of the modules and of their assemblies.

Thus, in industrial practice it is not possible to reliably pre-assembly the known modules prior to their packaging or prior to their introduction into a gelatin capsule, unless no gluing or welding or—at the very least—controlled wetting of their contact surfaces is carried out. However, with these methods, the practical assemblage of the modules is difficult to obtain on industrial scale, since no automatic machines exist capable to perform this task.

In addition, the modules prepared are compacts having porosity that is functional to substance release. This makes hardness and strength relatively low. Therefore, any not strictly necessary handling step constitutes an additional risk, and the modules require very careful manipulation during assemblage of two or more of them using ultrasound pressure or gluing wetting.

The aforementioned drawback is particularly pronounced in the production of the known assemblies resulting from concave/concave alignment and thus comprising an internal hollow inner space, which must be reliably sealed, such as to be air- and liquid-tight. This can currently not be achieved in a single operative step. Hence, a need exists for the provision of new release modules and of new assemblies, whose assemblage on industrial scale is substantially eased/improved and whose handling on industrial scale does not lead to disengagement of the single modules prior to their inclusion into gelatin shells or prior to final packaging. Preferably, the new release modules should provide an eased possibility to construct assemblies harboring a hollow inner space.

### SUMMARY OF THE INVENTION

The present invention therefore aims at resolving the aforementioned problems providing new modular systems for the controlled release of substances, characterized by eased industrial processing, in particular by the possibility of eased and long-lasting assemblage brought about by the simple aligning and mechanical engagement of the modules to provide new assemblies.

Thus, according to a first aspect, the present invention provides a module which is destroyed in the presence of water solution, composed of a compressed powdery mixture, the said powdery mixture comprising a matrix building component, suitable to release an optionally included active substance into a surrounding aqueous liquid, and one or more tabletting aids chosen from the group consisting of lubricants, glidants, and anti-adherent agents, the said module being provided with male topological features allowing its connection to a corresponding female module, the said male topological features comprising: a base (1) having a face (2) featuring an external perimeter (3); an annular protrusion (4) extending from said base (1) and having an inner (7) and an outer (5) lateral wall, said outer lateral wall (5) forming with said face an edge (6) completely contained within said external perimeter (3) and not coincident with it, said outer lateral wall (5) being adapted to engage by friction the wall of a cavity provided in the female module, such as to create a mating connection between the two modules, wherein, in the composition of the male module, the percentage ratio between the tabletting aid and the matrix building component is comprised between 1:2.5 and 1:999.

According to a second aspect, the present invention provides a module which is destroyed in the presence of water solution, composed of a compressed powdery mixture, the said powdery mixture comprising a matrix building component, suitable to release an optionally included active substance into a surrounding aqueous liquid, and one or more tabletting aids chosen from the group consisting of lubricants, glidants, and anti-adherent agents, the said module being provided with male topological features allowing its connection to a corresponding female module, the said male topological features comprising: a base (1) having a face (13) featuring an external perimeter (3); a protrusion (14) extending from said base (1) and having a straight lateral wall (15), said straight lateral wall (15) forming with said face an edge (6) completely contained within said external perimeter (3) and not coincident with it, said straight lateral wall (15) being adapted to engage by friction the wall of a cavity provided in the female module, such as to create a mating connection between the two modules, wherein, in the composition of the male module, the percentage ratio between the tabletting aid and the matrix building component is comprised between 1:2.5 and 1:999.

The percentage ratio means the ratio between the weight percentage of tabletting aid included into the formulation and the weight percentage of matrix building component included into the formulation. The present invention also provides for an assemblage kit comprising at least one male module (either according to the first or according to the second aspect of the invention) and at least one corresponding female module. The assembly of one male module, according to one of the embodiments of the present invention, is obtained by the frictional mechanical engagement to one corresponding female module. These connected modules can be part of a pile made by connecting over them additional female modules.

The modules can be also directly assembled inside a gelatin capsule. The pile formed in the capsule is firmly assembled by the pressure provided during the capsule closure.

The present invention also provides for the coating of the assembled modules by applying on the assembled system a film of polymeric substance or a layer of coating products.

Additionally, the present invention provides for a powdery composition employable in the construction by powder compression, extrusion or molding injection of the male and female modules, providing a process for obtaining the modules according to the present invention.

### DESCRIPTION OF THE FIGURES

The invention described and claimed in the present patent application is further illustrated, without limiting the same, in the enclosed Figures, which refer to some non-exhaustive examples of the possible embodiments of the invention and in which:

FIG. 1 is a lateral view of a male module according to the first embodiment of the present invention.

FIG. 2 is a cross-sectional view of the male module according to FIG. 1.

FIG. 3 is a cross-sectional view of another male module according to both of the embodiments of the present invention.

FIG. 4 is a cross-sectional view of still another male module according to both embodiments of the invention.

FIG. 5 is a partial cross section of the male module of FIG. 4.

FIG. 6 is a lateral view of a female module, connectable with a male module of the present invention.

FIG. 7 is a cross-sectional view of the female module according to FIG. 6.

FIG. 8 is a lateral view of a male module according to the second embodiment of the present invention.

FIG. 9 is a cross-sectional view of the male module according to FIG. 8. As one can see, the depicted male module bears also female features, making the same connectable with another male module of the present invention.

FIG. 10 is a cross-sectional view of an assembly according to the present invention comprising a male module according to both embodiments of the present invention (as per FIG. 4) as well as a male module according to the present invention (FIG. 8) connected thereto.

FIG. 11 is a cross-sectional view of an assembly according to the present invention comprising two male modules according to the second embodiment of the present invention (two modules as per FIG. 8).

FIG. 12 is a cross-sectional view of an assembly according to the present invention comprising one male module according to both embodiments of the present invention (as per FIG. 4) and two male modules according to the second embodiment of the present invention (two modules as per FIG. 8).

### DETAILED DESCRIPTION OF THE INVENTION

With the aim of overcoming the above-identified drawbacks, the inventors of the present Application have discovered a way to obtain new assembly modules employable for manufacturing adaptable modified release systems, whose assemblage and handling on industrial scale is markedly improved. In particular, the goal of avoiding the inadvertent disassembly of already combined modules in condition of industrial handling has been attained through the provision of an improved topology, compared to WO 03/043601, namely allowing for a lasting, purely mechanical engagement despite the module are powdery and porous since they are manufactured by powder compression.

The new mechanical way of module assembling was not previously considered applicable to modules that are porous and relatively strong compact of powders. On the contrary, despite the individual modules are porous compacts with moderate hardness, we discovered the possibility to assemble two or more modules in one solid piece or system by a technique defined snap, click or friction interlocking performed thanks to specially designed parts of the modules. This is obtained by means of the appropriate shaping in correspondence of the fitting area of the modules manufactured using powdered particulate materials. Thus, the invention consists in a new system for the controlled release of substance manufactured with the innovative concept of fitting together porous and powdery pieces having complementary shape by snap, click or friction fitting. The achievement is due to the fact that the piece composition introduces enough resistance in the module strength, and in particular in protruding portions of it, to allow the modules to bear the typical snap or click mechanism required for fitting modules together in a system.

To that respect, the new modules bear, for the first time, porous and powdery male topological features allowing connection of the said modules to a corresponding female module, the said male topological features comprising, according to a first embodiment: a base (1) having a face (2) featuring an external perimeter (3); an annular protrusion (4) extending from said base (1) and having an inner (7) lateral wall and an outer straight (5) lateral wall, said outer straight lateral wall (5) forming with said face an edge (6) completely contained within said external perimeter (3) and not coincident with it, said outer straight lateral wall (5) being adapted to frictionally engage the wall of a cavity provided in the female module.

According to a preferred variant of the said first embodiment, the face (2) of the male modules according to the first embodiment of the present invention presents a cavity (8). Particularly preferred are male modules of the aforementioned type wherein said cavity (8) has an internal wall (9), which is flush with the inner wall (7) of said annular protrusion (4).

With respect to the features (1)-(7) identified above, which are associated with the particular male topology present on the modules according to the first embodiment of the present invention, it is noted that base (1) can be of any adequate polyhedric shape, though a cylindrical shape is normally preferred. Face (2) present on base (1) can be of any adequate polygonal shape, though a circular shape is preferred. It is also noted that while it is preferred that face (2) is planar or at least substantially planar, it can also comprise deviations from planarity, e.g., face (2) can present a cavity (8). Depending on the shape of face (2), perimeter (3) can be polygonal or circular. As regards annular protrusion (4), the same can also be either polygonal or circular. While it is preferred that annular protrusion (4) is continuous, it can be, nevertheless also interrupted at some points of its perimeter. In this case, annular protrusion (4) is configured as a “crown” of teeth protruding from face (2). As regards the outer straight lateral wall (5) of the said annular protrusion (4), while it is preferred that it forms a right angle with face (2), it can nevertheless also deviate from a right angle, in particular be comprised between 85° and 95°, as long as the resulting shape still allows for the mating engagement with a correspondent female module. As regards edge (6) it is a part of face (2) and while it is preferred that edge (6) is planar, it can also be substantially planar. As regards inner wall (7), while it is preferred that it forms a right angle with face (2), it can nevertheless also deviate from a right angle, as it does not directly interact, during engagement, with the female module. Nevertheless, it can contribute to ease of engagement, e.g. if the inner wall (7) is tilted in such a way with respect to face (2) that the resulting annular protrusion has a tapering cross-section. In this case, outer straight wall (5) and inner wall (6) are not strictly parallel.

According to a second embodiment of the present invention, the new modules bear, for the first time, porous and powdery male topological features allowing connection of the said modules to a corresponding female module, the said male topological features comprising: a base (1) having a face (13) featuring an external perimeter (3); a clindrical protrusion (14) extending from said base (1) and having a straight lateral wall (15), said straight lateral wall (15) forming with said face (13) an edge (6) completely contained within said external perimeter (3) and not coincident with it, said straight lateral wall (15) being adapted to engage by friction the wall of a cavity provided in the female module. According to a preferred variant of the second embodiment of the invention, protrusion (14) features, at the end of straight lateral wall (15), a tapering extremity (16) to ease insertion into the female module.

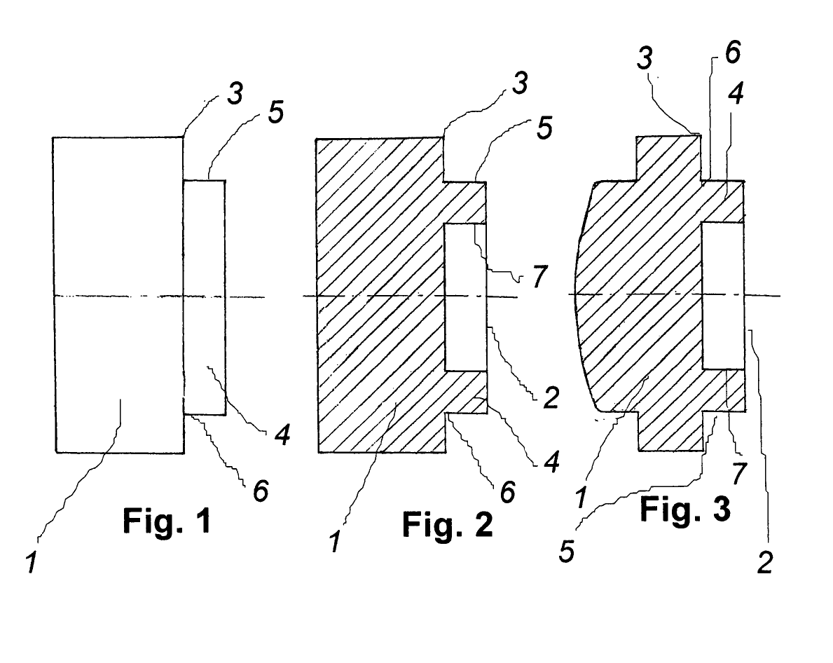

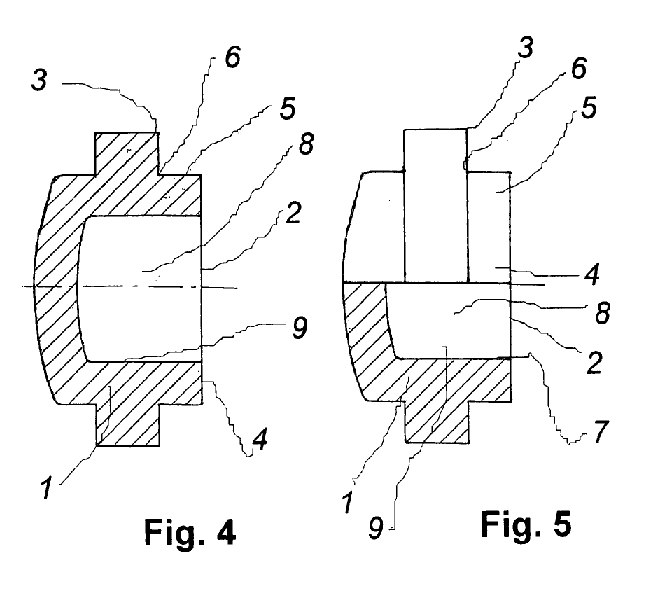

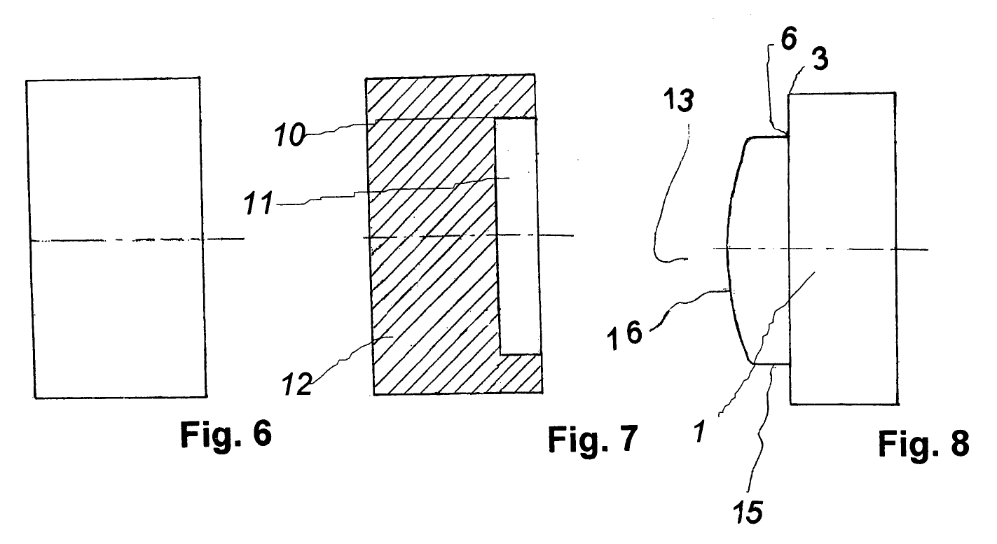

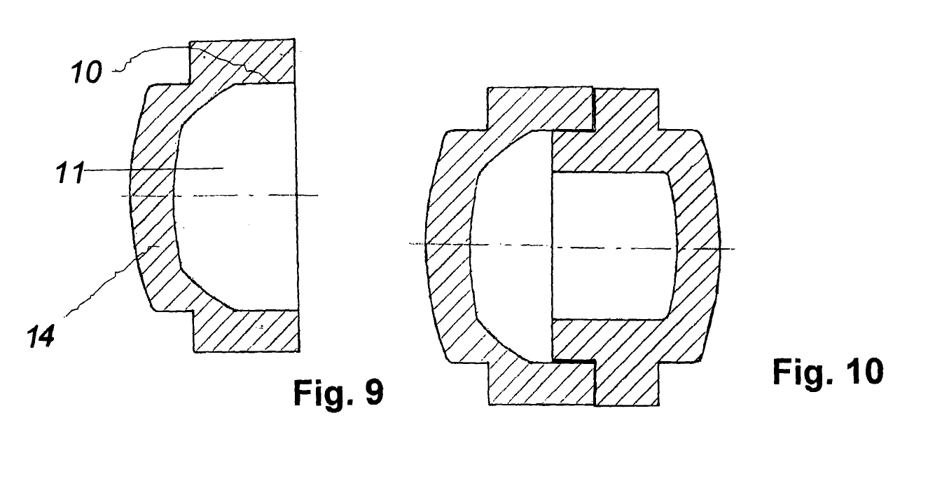

With respect to the features (1), (3), (6), (13), (14), (15) identified above, which are associated with the particular male topology present on the modules according to the second embodiment of the present invention, it is noted that base (1) can be of any adequate polyhedric shape, though a cylindrical shape is normally preferred. Face (13) present on base (1) can be of any adequate polygonal shape, though a circular shape is preferred. Depending on the shape of face (13), perimeter (3) can be polygonal or circular. As regards protrusion (14), the same can also be either polygonal or circular. As regards straight lateral wall (15) of the said protrusion (14), it is preferred that it forms a right angle with edge (6). According to a less preferred embodiment, the angle between straight lateral wall (15) and edge (6) can also slightly deviate from a right angle, in particular be comprised between 88° and 92°, as long as the resulting shape still allows for the mating engagement with a correspondent female module. As regards edge (6) it is a part of face (13) and while it is preferred that edge (6) is planar, it can also be substantially planar. Adequate female modules employable with the modules according to the present invention can feature a body (12) presenting a cavity (11) with lateral walls (10). It thus appears that modules according to the present invention are modules displaying at least one set of male topological features according to the first or the second embodiment of the invention. For example, FIG. 1 shows a module of the invention displaying the male topological features according to the first embodiment. FIG. 3 shows another module of the invention which features, on a first side of its base (1) male topological features according to the first embodiment of the invention and—on the second side of its base (1)—male topological features according to the second embodiment of the present invention. This module is thus a “mixed bimale”. FIG. 4 is another, alternative example of such a “mixed bimale”. While not specifically shown in the Figures, also “homogeneous bimales” of both of the embodiments are contemplated by the present invention. Also FIG. 9 it shows the invention, seen that the module depicted therein features on a first side of its base (1) male topological features according to the second embodiment of the invention. Instead, the second side of its base (1) features female topological features. The module is thus a “male/female” one, whose male part is shaped according to the second embodiment. While not specifically shown in the Figures, also “male/female” moduli whose male part is shaped according to the first embodiment are contemplated by the present invention.

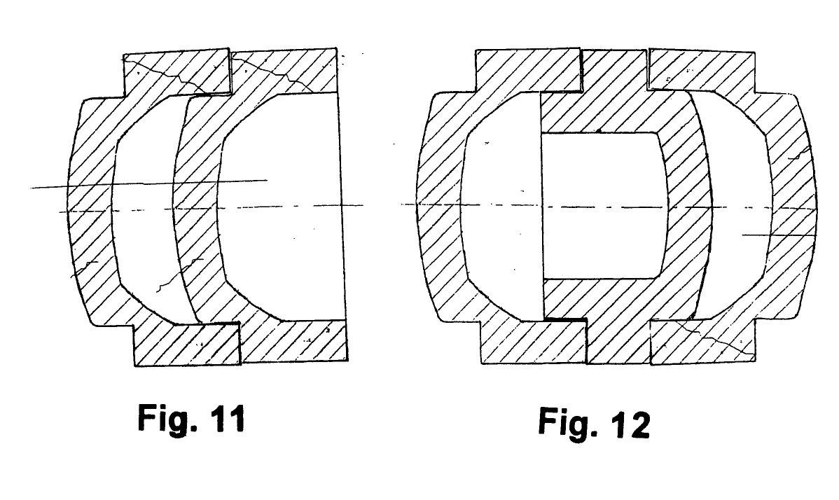

Assemblies according to the present invention are assemblies comprising modules of the present invention. FIG. 10 shows an example of such an assembly, comprising a “mixed bimale” module which is assembled with a “male/female” module according to the second embodiment. FIG. 11 shows another example, namely an assembly obtained from two “male/female” moduli, whose male part is shaped according to the second embodiment. FIG. 12 shows still another example, wherein a further “male/female” module according to the second embodiment of the present invention has been assembled with an assembly as per FIG. 10, for example, on the “mixed bimale” module comprised by it.

It is thus apparent how an assemblage kit comprising one or more of the male modules of the present invention, allows by assembling the modules between each other and/or with corresponding female modules, the construction of assemblies of various types.

As is apparent from the above, attainment of a well defined geometry with adequately narrow tolerances is important for ensuring constant quality of the two particular male topologies of the modules according to the two embodiments of the invention herein described. Tiny protrusions were however never present, so far, in objects made from compressed powders, as e.g. pharmaceutical tablets which normally bear at most incisions reproducing the name of the product or of the producer. In addition, the assembly by frictional engagement of tiny protrusions has never been done before on tablets mainly due to the relative strength of these products that are porous and relatively friable. In particular, so far, such task has never been achieved with pharmaceutical formulations as these are not only of limited size (they must be “administrable” to patients, in particular “swallowable”) but on top of that the employable ingredients are limited to the substance conventionally employed in the manufacture of tablets. As is known, tablets are bodies of compacted powders which have an inherent porosity and friability which limits the enlargement of their outer surface, due to the susceptibility of the exposed, newly created surfaces to break at the mechanical loads arising during the industrial handling. The former is true especially for convex, protruding surfaces like the male topologies here at issue. Indeed, as is known, conventional tablets have normally comparatively simple shapes with low surface area/volume ratios and only in exceptional cases, with grooves allowing for subdivision into halves for purposes of dosage adjustment.

Moreover convex, protruding surfaces are generated by corresponding concave configurations of the tableting punches, whose difficulty of clearance after the compression step increases with the sophistication of the imparted topology. It is therefore important to underscore that, despite the herein described modules are porous compacts of powder and have the typical hardness and friability of products obtained by powder compression, due to the particular composition and the particular topology of fitting surfaces herein described, the modules provided by the present invention resist the fitting operation and maintain the system firmly assembled. They are thus fit for industrial handling. In particular, using the friability apparatus and the procedure described in the EU Pharmacopoeia (5^{th} Edition (Ph.Eur. 5)), the strength of the modules can be checked. In particular, friability testing measures the weight loss by the modules simulating their resistance during the process of coating, transportation, assembly or packing. This weight loss due to frictional abrasion or detachment of protuberances or edges of the modules must be less than 1%, a condition which is fulfilled by the modules of the present invention.

According to a second aspect, the present invention also provides for an assemblage kit comprising at least one male module according to the present invention and at least one corresponding female module.

According to a third aspect, the present invention also provides for an assembly comprising at least one male module, according to the present invention, connected by frictional mechanical engagement to at least one corresponding female module. These connected modules (“assemblies”) can be part of a pile built by connecting over them additional female modules (or male/female modules) staked on the firstly connected male or “bimale” modules. It is an important feature of the assemblies of the present invention that, once they have been assembled, they do not disengage in conditions of the friability testing according to the <<Friability of uncoated tablets>> test in European Pharmacopoeia 5^{th} Edition (Ph.Eur. 5).

According to a fourth aspect, the present invention also provides for the modules directly assembled inside a gelatin capsule or in a polymeric cylinder. In this aspect, the modules are individually introduced in the body of a capsule or in the polymeric tube in the appropriate sequence and in the position and direction suggested for the male/female engagement or additional female modules stacking. The pile formed in the capsule is firmly assembled by the pressure provided during the capsule closure after the insertion of the capsule cap on the body containing the positioned modules.

According to a fifth aspect, the present invention also provides for the coating of the assembled modules in a rotating pan or in a fluid bed apparatus, obtained by applying on the assembled system a film of polymeric substance or a layer of coating products, (minerals, starches, sugars) with the aim to protect, control or delay the release of the coated substances.

Additionally, the present invention provides for a powdery composition employable in the construction by powder compression of the male and female modules according to the present invention.

Further, the present invention also provides a process for obtaining the modules according to the present invention, comprising the optional mixing of an active substance with the herein described powdery composition, compression of the powdery composition, be it loaded with active substance or not, within a tableting machine or molding press of common use in pharmaceutical industry, employing particularly shaped punches, capable of generating, on the compressed module, the particular male topological features herein described, and recovery of the thereby obtained modules from the tableting machine or press.

Therefore, the provision of adequate powdery mixtures to be employed in the construction of the herein described modules is a further achievement of the present invention. In particular, the powdery mixture must have two characteristics clearly antagonist: it must be highly cohesive for obtaining the hardness appropriated for the firm engagement of male and female surfaces, but also sufficiently anti-adherent to avoid the sticking on the surface of the tools in particular in correspondence of the concavities, so allowing the recovery of the compact. Therefore, it is compulsory to find an appropriate balance between the substances capable to give hardness avoiding sticking and, on the other hand, lubrication or anti-adhesion avoiding weakening. In particular, the present invention employs a powdery mixture which comprises one or more matrix building components, suitable to bind and release into a surrounding aqueous liquid an optionally included active substance, and one or more tableting aids chosen from the group consisting of lubricants, glidants, and anti-adherent agents.

Applicants have found that with the herein described male geometries of the first and the second embodiments of the present invention, it is necessary to employ particular powdery mixtures to ensure, on the one hand, a Pharmacopoea-conform weight loss of the non-assembled modules during friability testing, and, on the other hand, protection against disengagement of assemblies comprising modules according to the present invention.

In particular it has been found that the modules according to the present invention, can be obtained employing standard equipment for the compaction of powders (in particular the tabletting machines employed in pharmaceutical industry) if the percentage ratio between the tableting aid and the matrix building component is comprised between 1:2.5 and 1:999. Preferably, the percentage ratio between the tableting aid and the matrix building component is comprised between 1:2.5 and 1:180, more preferably between 1:3 and 1:120. Most preferably, it is comprised between 1:4 and 1:60. For example, according to a preferred embodiment, the weight percentages of the tableting agent have to be comprised between 0.5% and 10% and the weight percentages of the matrix building component have to be comprised between 25% and 90%, the remainder up to 100% comprising an active principle.

The term matrix building component embraces substances used for binding particles and at the same time for controlling the delivery of active principles, such as cellulose derivatives, syntethic and semisynthetic polymers, sugars as glucose and saccharose, polysaccarides and proteins. Particularly preferred are hydroxypropylmethilcellulose, hydroxypropylcellulose, hydroxyethylcellulose, microcrystalline cellulose, carboximethylcellulose, methylcellulose, cellulose acetate, cellulose acetate propionate, ethyhlcellulose poliox, granular calcium phosphate, sucrose, glucose, scleroglucan, alginates, chitosan, cyclodextrins. pectins, arabic gum, guar gum, carrageenan, xantan gum, acacia, starch, dextrins, gelatin, collagen, albumin, polyvinylpyrrolidone, polymethacylates, carbomer, poloxamer, polyethylene glycol, polyvinyl alcohol, polyethylene oxide. As regards control of delivery, it appears from above that (and it is intended that) the nature of the matrix building components is such that the modules obtained there from are destroyed quickly or slowly upon exposure to water solution. According to the intended use, i.e. the provision of an agrochemical, nutraceutical or pharmaceutical formulation, the matrix building components can thus be adequately chosen by the skilled man to allow for destruction of the modules upon interaction with aqueous fluids within a pre-envisaged time interval.

The term tableting aids refers to lubricants, glidants and anti-adherent agents, i.e. those pharmaceutically acceptable additives which are commonly employed to allow the filling of the powdery mixture into the tabletting press and the extraction of the compacted body from the dies. Tableting aids are widely known in the art and employed on the one hand, to increase the flowability of the powder and, on the other hand, to reduce surface adhesion between the compacted body and the punches. Prominent, but not exclusive examples of tableting aids include calcium stearate, glyceryl monostearate, glyceryl palmitostearate, hydrogenate castor oil, hydrogenate vegetable oil, light mineral oil, magnesium stearate, colloidal silica, sodium benzoate, sodium lauryl sulphate, sodium stearyl fumatare, stearic acid, talc, zinc stearate.

In the herein described modules, further to the matrix building component and to the tableting aids also active principles can be employed.

Modules bare of active principles are interesting as so-called “auxiliary modules” in the sense of WO 03/043601, as the said auxiliary modules nevertheless influence the release profile of the active principle contained in a distinct, further module which with they can be combined to make up an assembly or, in the alternative, of active principle contained in a hollow space obtainable by assembling two modules (see e.g. the “floating” module of FIG. 10, whose hollow inner space can also be charged with an active principle or a formulation thereof). Moreover, auxiliary modules, used to engage one or more modules, after the contact whit water or gastric juice break and release one or more pieces of the assembled system in order to control the release of drugs with different kinetics.

As regards instead the “non-auxiliary” modules of the present invention, they can contain a wide range of active principles, be it for pharmaceutical, neutraceutical or agricultural application. Prominent but not exclusive examples of employable active principles include:
human and veterinary drugs (analgesics, antibiotics, antiviral drugs, drugs for central nervous system, hypnotics and sedatives, diuretics, anticancer drugs, hormones, hormones antagonists, antihyperlipemics, antimycotics, prokinetic agents, antiemetics, immunomodulators, drugs affecting renal and cardiovascular function)
diagnostics and biologically active compounds
herbal medications, homeopathic drugs and compounds related to alternative medicines
nutraceuticals and phytotherapeutics (vitamins, co-enzymes, amminoacids, minerals, peptides, carbohydrates, lipids)
substances for plants and environment (pesticides, growth factors, phytohormones, anti-pollutants, fertilizers)

The modules of the present invention may also contain further excipients like e.g. diluents, adsorbents, buffers, antioxidants, flavoring agents, wetting agents, emulsifying agents, dyes, preservatives and others.

A further object of the present invention is a process for obtaining the herein described male modules comprising the optional mixing of an active substance with the powdery composition, compression of the powdery composition, be it loaded with active substance or not, within a tableting machine or press of common use in pharmaceutical industry, employing particularly shaped punches, capable of generating, on the compressed module, the male topological features herein described, and removal of the thereby obtained modules from the tableting machine or press. It is thus an advantage of the present invention that apart from the employment of particularly shaped punches, the tableting process herein used is perfectly conventional.

### EXAMPLES

### Example 1

The example describes the manufacture and assembling of release modules containing riboflavin according to the present invention in order to provide a floating “controlled release” system. This kind of assembly is composed of two modules, containing 1.3 mg of riboflavin each. The particular assembly employed here is the one depicted in FIG. 10, i.e. being composed of a “mixed bimale” module according to both embodiments of the invention and a “male/female” according to the second embodiment of the present invention.

The unitary formula for preparing 500 “mixed bimale modules” and 500 male/female modules, was the following:

| | ×1000 | |
|---|---|---|
| | MODULES | |
| COMPONENTS | (g) | % |
| Riboflavin | 1.5 | 1.19 |
| Hydroxypropylmethylcellulose | 50.0 | 39.84 |
| Lactose | 29 | 23.10 |
| Microcrystalline cellulose | 29 | 23.10 |
| Polyethylenglycol 6000 | 6.0 | 4.78 |
| Polyvinylpirrolidone | 6.0 | 4.78 |
| Talc | 3.5 | 2.79 |
| Magnesium stearate | 0.5 | 0.40 |

### a) Preparation and Mixing of the Components

The powders of all the different components were firstly sieved in order to break the clumps. Powders were then mixed together in a Turbula® apparatus for about 30 minutes. The ratio of percentages between tableting aids and matrix building percentages was 1:30.

### b) Compression

Modules were produced by direct compression of mixed powders using an alternative tablet press equipped with special shaped punches (different sets for the two types of modules employed here) with diameter 7.4 mm. Compression force was set between 20-35 KN. The weight of each module was 125.5±5 mg. Friability was assessed according to the <<Friability of uncoated tablets>> test in European Pharmacopoeia 5^{th} Edition (Ph.Eur. 5). The loss of mass of both types of modules due to the mechanical stress was lower than 1%. “Bimale and “male/female” modules were assembled by aligning the female part of the “male/female” module with the male topology according to the first aspect of the present invention present on the “bimale” module and application of a compression force of 10 N (see FIG. 10).

The so-obtained assemblies were tested as to their tendency to disengage during industrial handling. To that respect, the mechanical resistance of the assemblies was tested with a Tablet Friability Apparatus described in Ph.Eur. 5. 20 assemblies were placed in the rotating drum of the apparatus (25 rpm) for at least 4 minutes. Typical mechanical stresses experienced by assemblies during rolling were impact and friction with drum walls and between assemblies. At the end of the test, assemblies were recovered and the percentage of assemblies broken up evaluated. None of the assemblies disengaged or broke apart.

### Example 2

### Comparative

This example describes the manufacture and assembling of male/female modules (with male features according to the first embodiment) as per FIG. 9, containing 5 mg of lycopene. They are intended for the preparation of assembled systems composed of two staked modules, as shown in FIG. 11.

The unitary formula for preparing 500 male/female modules was the following:

| | ×1000 | |
|---|---|---|
| | MODULES | |
| COMPONENTS | (g) | % |
| Lycopene 5% w/w | 111.75 | 86.29 |
| Microcrystalline cellulose | 11.75 | 9.08 |
| Sodium Lauryl Sulphate | 1.0 | 0.77 |
| Talc | 4.0 | 3.09 |
| Magnesium stearate | 1.0 | 0.77 |

### a) Preparation and Mixing of the Components

The powder components were then mixed together in a Turbula® for about 30 minutes. The percentage ratio between tableting aids and matrix building components was 1:1.96.

### b) Compression

Modules were produced by direct compression of mixed powders using an alternative tablet press, using special shaped punches with diameter 7.4 mm and compression forces estimated in 20-30 KN. The weight of each module was 129.5±5 mg. Friability was assessed according to the <<Friability of uncoated tablets>> test in European Pharmacopoeia 5^{th} Edition (Ph.Eur. 5). The loss of mass of modules due to the mechanical stress was below 1%.

By stacking and applying a compression force of 10 N, assemblies of two modules were created. Assemblies of modules were tested as to their tendency to disengage. The mechanical resistance of the assemblies was tested with a Tablet Friability Apparatus described in Ph.Eur. 5. 20 assemblies were placed in the rotating drum of the apparatus (25 rpm) for at least 4 minutes. At the end of the test, assemblies were recovered and the percentage of assemblies broken up evaluated. In this experiment several of the assemblies disengaged/broke apart.

### Example 3

The example describes the manufacture and assembling of modules containing 80 mg of clyndamicin for preparation of a floating system for substance release as per FIG. 10.

The unitary formula for preparing 500 “bimale” and 500 “male/female” modules (the geometries involved were the same as in example 1), was the following:

| | ×1000 | |
|---|---|---|
| | MODULES | |
| COMPONENTS | (g) | % |
| Clyndamicin | 80.0 | 68.95 |
| Polyvinylpirrolidone | 3 | 2.55 |
| Hydroxypropylmethylcellulose | 27.0 | 23.27 |
| Talc | 5.38 | 4.63 |
| Magnesium stearate | 0.7 | 0.60 |

### a) Mixing of the Components

Clyndamicin and HPMC powders were granulated using a hydroalcoholic solution of polyvinylpirrolidone. The mixture was forced through a net of 0.5 mm. The granules were mixed with talc and magnesium stearate in a Turbula® apparatus for about 30 minutes. The percentage ratio between tableting aids and matrix building components was 1:4.9

### b) Compression

Modules were produced by direct compression of the mixed powders using an alternative tablet press, using special shaped punches (different sets for the two types of modules employed here) with diameter 7.4 mm and compression forces estimated in 20-30 KN. The weight of each module was 116±5 mg. Friability was assessed according to the <<Friability of uncoated tablets>> test in European Pharmacopoeia 5^{th} Edition (Ph.Eur. 5). The loss of mass of both types of modules, due to the mechanical stress was below 1%.

“Bimale and “male/female” modules were assembled by aligning the female part of the “male/female” module with the male topology according to the first aspect of the present invention present on the “bimale” module and application of a compression force of 10 N (see FIG. 10).

The assembled modules were floating in water.

The assemblies were tested as to their tendency to disengage during handling. The mechanical resistance of the assemblies was tested with a Tablet Friability Apparatus described in Ph.Eur. 5. 20 assembled delivery systems were placed in the rotating drum of the apparatus (25 rpm) for at least 4 minutes. At the end of the test, assemblies were recovered and the percentage of assemblies broken up evaluated. None of the assemblies disengaged/broke apart.

### Example 4

### Comparative

The example describes the manufacture of modules containing 125 mg of clyndamicin for a floating system as per FIG. 10 for substance release. The unitary formula for preparing 500 “bimale” and 500 “male/female” modules, was the following:

| | ×1000 | |
|---|---|---|
| | MODULES | |
| COMPONENTS | (g) | % |
| Clyndamicin | 125.0 | 99.0 |
| Microcrystallinecellulose | 0.625 | 0.5 |
| Magnesium stearate | 0.625 | 0.5 |

### a) Mixing of the Components

The different components were mixed together in a Turbula® apparatus for about 30 minutes. The percentage ratio between the percentages of tableting aids and matrix building components was 1:1

### b) Compression

Modules were produced by direct compression of the mixed powders using an alternative tablet press and special shaped punches (different sets for the two types of modules employed here) with diameter 7.4 mm and compression forces estimated in 20-30 KN. The weight of each module was 126.5±5 mg. It was however impossible to obtain modules of both of the employed shapes, since the compressed material stuck to the punches of the tableting machine. This formulation at borderline of the ratio between the two categories of adjuvants was not tabletable with punches of the required topology.

### Example 5

The example describes the manufacture and assembling of “male/female” modules as per FIG. 9 containing 50 mg of artesunate for assembly multi-modules systems with the already assembled modules of clyndamicin of the example 3 system for substance release.

The unitary formula for preparing 500 “male/female” modules was the following:

| | ×1000 | |
|---|---|---|
| | MODULES | |
| COMPONENTS | (g) | % |
| Artesunate | 50.0 | 44.64 |
| Microcrystallinecellulose (Avicel) | 53.37 | 47.65 |
| Carboxymethilstarch | 4.48 | 4.01 |
| Talc | 3.36 | 3.00 |
| Magnesium stearate | 0.79 | 0.70 |

### a) Mixing of the Components

The powders of all the different components were mixed together in a Turbula® apparatus for about 30 minutes. The percentage ratio between tableting aids and matrix building components was 1:14

### b) Compression

Modules were produced by direct compression of the mixed powders using an alternative tablet press, using special shaped punches with diameter 7.4 mm and compression forces between 20-30 KN. The weight of each module was 112±5 mg. Friability was assessed according to the <<Friability of uncoated tablets>> test in European Pharmacopoeia 5^{th} Edition (Ph.Eur. 5). The loss of mass of the modules due to the mechanical stress was less than 1%.

“Male/female” modules as per FIG. 9 were stacked on the floating assemblies as per example 3, obtaining assemblies as per FIG. 11 applying a compression force of 10N.

Assembled modules were tested as to their tendency to disengage during handling. To that respect, the mechanical resistance of the assemblies was tested with a Tablet Friability Apparatus described in Ph.Eur. 5. 20 assembled delivery systems were placed in the rotating drum of the apparatus (25 rpm) for at least 4 minutes. At the end of the test, assemblies were recovered and the percentage of assemblies broken up evaluated. In the experiment performed none of the assemblies disengaged or broke apart.

Assemblies as per Examples 1, 3 and 5 can thus be employed to convey the contained active principles to the intended site of action.

1-28. (canceled)

29. A module which is destroyed in the presence of water solution, composed of a compressed powdery mixture, the said powdery mixture comprising:
a matrix building component, suitable to release an included active substance into a surrounding aqueous liquid, in an amount between 25 and 90 wt %,
one or more tabletting aids selected from the group consisting of lubricants, glidants, and anti-adherent agents, in an amount between 0.5 and 10 wt %,
the remainder up to 100 wt % being an active principle, the said module being provided with male topological features allowing the connection to a corresponding female topological featured base or face of a second module, the said male topological features comprising:
a base (1) having two opposed faces both featuring an external perimeter (3),
at least one annular protrusion (4, 14) extending from one of said faces of the base,
a cavity (8, 11) on said annular protrusion (4) or on the face opposed to the face of the annular protrusion (14),
said at least one annular protrusion (4, 14) having an outer straight lateral wall (5, 15) forming with the face concerned an edge (6) completely contained within said external perimeter (3) and not coincident with it, said outer straight lateral wall (5, 15) being adapted to engage by friction the wall of a cavity provided in the female base or face of said second module, such as to create a mating connection between said two modules.

30. The module according to claim 29, wherein, when the cavity (11) is on the face opposed to the face of the annular protrusion (14), said annular protrusion (14) features a tapering extremity (16).

31. The module according to claim 29, having two annular protrusions (4, 14), the first annular protrusion (4) extending from a first face of the base and the second annular protrusion (14) extending from the face opposed to the first one.

32. The module according to claim 29, wherein the active principle is selected from the group consisting of human and veterinary drugs, diagnostics and biologically active compounds, herbal medications, homeopathic drugs and compounds related to alternative medicines, nutraceuticals, phytotherapeutics and substances for plants and environment.

33. The module according to claim 29, wherein the percentage ratio between the tabletting aid and the matrix building component is comprised between 1:3 and 1:120.

34. The module according to claim 33, wherein the percentage ratio between the tabletting aid and the matrix building component is comprised between 1:4 and 1:60.

35. An assemblage kit comprising at least two modules according to claim 29.

36. The assemblage kit according to claim 35, wherein said at least two modules are connected by mechanical friction engagement.

37. The assemblage kit according to claim 35, wherein at least one of said at least two modules has two annular protrusions (4, 14), the first annular protrusion (4) extending from a first face of the base and the second annular protrusion (14) extending from the face opposed to the first one.

38. The assemblage kit according to claim 37, further comprising a third module which is destroyed in the presence of water solution, composed of a compressed powdery mixture, the said powdery mixture comprising:
a matrix building component, suitable to release an included active substance into a surrounding aqueous liquid, in an amount between 25 and 90 wt %,
one or more tabletting aids selected from the group consisting of lubricants, glidants, and anti-adherent agents, in an amount between 0.5 and 10 wt %,
the remainder up to 100 wt % being an active principle, the said third module being provided with male topological features allowing the connection to a corresponding female topological featured base or face of an additional module, the said male topological features comprising:
a base (1) having two opposed faces both featuring an external perimeter (3),
at least one annular protrusion (4, 14) extending from one of said faces of the base,
a cavity (8, 11) on said annular protrusion (4) or on the face opposed to the face of the annular protrusion (14),
said at least one annular protrusion (4, 14) having an outer straight lateral wall (5, 15) forming with the face concerned an edge (6) completely contained within said external perimeter (3) and not coincident with it, said outer straight lateral wall (5, 15) being adapted to engage by friction the wall of a cavity provided in the female base or face of said additional module, such as to create a mating connection between said third and additional modules.

39. A process for obtaining the module according to claim 29, comprising the steps of compressing the powdery mixture within a tabletting machine or a molding press, employing particularly shaped punches or dies, capable of generating the male topological features on the compressed module, and removing the thereby obtained module from the tabletting machine or molding press.
